(19) United States
(12) Patent Application Publication
(10) Pub. No.: US 2011/0009757 A1
(43) Pub. Date: Jan. 13, 2011

(54) BLOOD PRESSURE INFORMATION MEASUREMENT DEVICE

(75) Inventors: Yoshihiko SANO, Kyoto-shi (JP); Chisato UESAKA, Kyoto-shi (JP); Hiroyasu ARIGA, Kyoto-shi (JP)

Correspondence Address: Omron Healthcare Co. Ltd.;c/o Keating & Bennett, LLP, 1800 Alexander Bell Drive, Suite 200, Reston, VA, 20191, US

(73) Assignee: OMRON HEALTHCARE CO., LTD., Kyoto-shi (JP)

(21) Appl. No.: 12/884,225

(22) Filed: Sep. 17, 2010

Related U.S. Application Data
(63) Continuation of application No. PCT/JP2009/054020, filed on Mar. 4, 2009.

(30) Foreign Application Priority Data
Mar. 19, 2008 (JP) 2008-071818

(51) Int. Cl. A61B 5/022

(52) U.S. Cl. 600/499

(57) ABSTRACT

A sphygmomanometer separately includes a cuff accommodating an air bladder and a main body provided with an expanding/contracting mechanism arranged to expand and contract the air bladder, and the cuff includes a tubular cuff main body portion into which an upper arm is insertable and a gripping portion provided on an outer peripheral surface of the cuff main body portion. The cuff main body portion has a tightening belt wrapped around the outer side of the air bladder and a tightening length adjustment mechanism arranged to variably adjust a tightening length of the tightening belt over the upper arm. The tightening length adjustment mechanism includes a winding roller arranged to wind and feed the tightening belt, a geared motor arranged to drive and rotate the winding roller, and an electromagnetic brake arranged to apply a braking force to the winding roller. With such a configuration, in the sphygmomanometer in which the cuff and the main body are separated from each other, the cuff can be easily attached to a measurement site, and reliable winding of the cuff around the measurement site can be repeated in every measurement.

### BACKGROUND OF THE INVENTION

1. Field of the Invention

The present invention relates to a blood pressure information measurement device capable of measuring blood pressure information such as a blood pressure value, and more particularly to a blood pressure information measurement device separately including a cuff attached to a measurement site to be used, and a main body mounted on a surface to be used.

2. Description of the Related Art

Acquiring blood pressure information of a subject is highly important in terms of knowing a health condition of the subject. In recent years, it is not limited to acquiring a systolic blood pressure value (a maximum blood pressure value), a diastolic blood pressure value (a minimum blood pressure value), and the like, whose effectiveness has been widely recognized as a representative index of health management from the conventional art, and attempts have been made in capturing a change in a heart load and hardness of an artery by acquiring a pulse wave of the subject. A blood pressure information measurement device is a device for obtaining the index for health management based on the acquired blood pressure information, and further utilization is expected in fields of early detection and prevention, treatment, and the like of circulatory system diseases. It should be noted that the blood pressure information widely includes various information of the circulatory system such as the systolic blood pressure value, the diastolic blood pressure value, an average blood pressure value, a pulse wave, a pulse beat, and an AI (Augmentation Index) value.

In general, a cuff accommodating a fluid bag is utilized for measuring the blood pressure information. The cuff indicates a band-shaped structure having an inner cavity capable of being wound around part of a living body, which is utilized for measuring the blood pressure information by injecting a fluid such as gas and liquid into the inner cavity so as to expand and contract the fluid bag. For example, in a blood pressure information measurement device for measuring a blood pressure value such as the systolic blood pressure value and the diastolic blood pressure value (hereinafter, also simply referred to as the sphygmomanometer), the cuff accommodating the fluid bag for compressing an artery is wound around a body surface of the living body, the wound fluid bag is expanded and contracted so as to capture an arterial blood pressure pulse wave as a change in the internal pressure of the fluid bag, thereby measuring the blood pressure value. It should be noted that the cuff particularly wound around an arm to be used is also called an arm band or a manchette.

The above sphygmomanometer is roughly divided based on a configuration difference into a sphygmomanometer in which the cuff and a main body are integrated, and a sphygmomanometer in which the cuff and the main body are separated from each other. In the sphygmomanometer in which the cuff and the main body are integrated, a hollow opening portion into which a measurement site is to be inserted is formed in the main body, and the cuff is provided with the main body so as to surround this hollow opening portion. Meanwhile, in the sphygmomanometer in which the cuff and the main body are separated from each other, an expanding/contracting mechanism for expanding and contracting an air bladder serving as the fluid bag accommodated in the cuff (normally a pressurization pump, an exhaust valve, or the like) is provided in the main body, and the air bladder accommodated in the cuff and the expanding/contracting mechanism provided in the main body are connected to each other by a flexible air tube.

For example, Japanese Unexamined Patent Publication No. 2005-237427 discloses a sphygmomanometer in which the cuff and the main body are integrated. In the sphygmomanometer in which the cuff and the main body are integrated, which is disclosed in Japanese Unexamined Patent Publication No. 2005-237427, a drive mechanism and the like for winding the cuff can be relatively easily provided in the main body with which the cuff is provided. Therefore, only by inserting the measurement site into the hollow opening portion provided in the main body, attachment of the cuff and measurement of the blood pressure value can be automatically performed. Thus, it can be said that the sphygmomanometer in which the cuff and the main body are integrated is excellent in that the cuff can be highly easily attached and detached. On the other hand, in the sphygmomanometer in which the cuff and the main body are integrated, there are problems including a problem that the device is necessarily increased in size and a problem that a measurement posture is limited to a specific posture. It cannot always be said that the sphygmomanometer is excellent in usability under any use environments.

Meanwhile, in the sphygmomanometer in which the cuff and the main body are separated from each other, by separating the cuff from the main body, the cuff can be relatively small. Therefore, the sphygmomanometer in which the cuff and the main body are separated has favorable usability under various use environments, and a degree of freedom in the measurement posture is significantly high in comparison to the sphygmomanometer in which the cuff and the main body are integrated. It can be said that the sphygmomanometer is also excellent in usability in this point. However, in the sphygmomanometer in which the cuff and the main body are separated from each other, an attachment task of the cuff is generally given into the hands of a human such as the subject. Thus, reliable winding of the cuff around the measurement site is not always repeated in every measurement. In order to more precisely and stably measure the blood pressure value, the cuff is required to be reliably wound around the measurement site. In this regard, the sphygmomanometer in which the cuff and the main body are separated still has room for improvement.

Therefore, variously formed cuffs have been conventionally proposed for reliably winding the cuff around the measurement site with favorable repetition in the sphygmomanometer in which the cuff and the main body are separated from each other. For example, Japanese Unexamined Patent Publication No. S61-238229 and Japanese Unexamined Patent Publication No. 2002-209858 and the like disclose a cuff accommodating a flexible member called a curler inside in addition to the air bladder. The curler is accommodated inside the cuff for maintaining an annular form of the cuff. The curler is annularly wrapped around the outer side of the air bladder and arranged inside the cuff, so that the cuff is formed to be elastically deformable in the radial direction. In the cuff provided with such a curler, the air bladder is fixed while being pushed toward the measurement site by the curler with proper pressing force after attachment. Thus, reliable fixation of the air bladder to the measurement site is repeated.

However, in the cuff accommodating the curler, there is a problem that a winding task thereof is troublesome in comparison to the above sphygmomanometer in which the cuff and the main body are integrated. This is due to the fact that the curler is shaped so that the curler during a time of non-attachment has a more reduced diameter than the measurement site in order to reliably press the air bladder onto the measurement site during a time of attaching the cuff. That is, during a time of attaching the cuff, the curler in a reduced diameter state is required to be once pushed and extended so as to be attached to the measurement site. This pushing and extending task is a cause of problems. Particularly, in a sphygmomanometer for domestic use, the subject himself/herself is required to wind the cuff around one of his/her arms. Thus, the subject is only able to use the other hand at a time of attachment. Therefore, the subject is required to get used to, to some extent, a task of pushing and extending the cuff in the reduced diameter state and attaching the cuff to the arm with a single hand.

In order to further easily attach the cuff, Japanese Unexamined Patent Publication No. 2006-68318 discloses a configuration of a cuff attachable to and detachable from the measurement site with a single touch operation. In the cuff disclosed in Japanese Unexamined Patent Publication No. 2006-68318, an elastic member such as a bias spring and a power transmission mechanism such as a slider are built inside the cuff so that size of the cuff in the radial direction is variable in connection with an operation of a user. Thereby, the cuff can be attached and detached with the single touch operation.

However, in the above cuff disclosed in Japanese Unexamined Patent Publication No. 2006-68318, although the cuff can be attached and detached with the single touch operation, a bias force for pushing the air bladder to the measurement site depends only on elastic force of the bias spring. Thus, there is a fear that a sufficient bias force is not obtained. In order to obtain a larger bias force, it is thought that a bias spring having a larger spring constant is utilized. However, in that case, since a force required for an operation of an operation unit becomes larger, there is a problem that operability is lowered. Since configurations of the above-described power transmission mechanism and the like are complicated, there is also a problem that the power transmission mechanism is easily broken due to repetitive use.

### SUMMARY OF THE INVENTION

Therefore, in view of solving the above problems, preferred embodiments of the present invention provide a cuff of a sphygmomanometer that is easily attached to a measurement site and in which the cuff and a main body are separated from each other, and reliable winding of the cuff around the measurement site is easily repeated in every measurement.

A blood pressure information measurement device according to a preferred embodiment of the present invention separately includes a cuff attached to a measurement site to be used during a time of measurement, and a main body mounted or supported on a surface to be used during a time of measurement. The cuff includes a tubular cuff main body portion including a hollow opening portion into which the measurement site is insertable in the axial direction, and a gripping portion provided on an outer peripheral surface of the cuff main body portion. The cuff main body portion has a fluid bag arranged to compress the measurement site, a tightening belt wrapped around the outer side of the fluid bag, and a tightening length adjustment mechanism arranged to variably adjust a tightening length of the tightening belt over the measurement site. The main body includes an expanding/contracting mechanism arranged to expand and contract the fluid bag. The tightening length adjustment mechanism includes a winding roller arranged to wind and feed the tightening belt, an electric motor arranged to drive and rotate the winding roller in the forward direction and the backward direction, and a brake arranged to apply a braking force to the winding roller at a time of stopping the electric motor.

In the blood pressure information measurement device according to a preferred embodiment of the present invention, the gripping portion preferably includes a base portion attached to the cuff main body portion, and in that case, the tightening length adjustment mechanism is preferably accommodated in the base portion.

In the blood pressure information measurement device according to a preferred embodiment of the present invention, a winding operation unit arranged to receive a command to start a winding operation of the tightening belt by the winding roller is preferably provided in the gripping portion, and in that case, it is more favorable that the winding operation unit is defined by a push button.

In the blood pressure information measurement device according to a preferred embodiment of the present invention preferably further includes a tightening force detection mechanism arranged to detect a tightening force of the tightening belt over the measurement site. In that case, the tightening length adjustment mechanism preferably stops a winding operation of the tightening belt by the winding roller when the tightening force detection mechanism detects a predetermined magnitude of the tightening force.

In the blood pressure information measurement device according to a preferred embodiment of the present invention, the tightening force detection mechanism preferably detects an internal pressure of the fluid bag at a time of the winding operation of the tightening belt by the winding roller in a state where a predetermined amount of fluid is injected into the fluid bag by the expanding/contracting mechanism, thereby detecting the tightening force of the tightening belt over the measurement site.

In the blood pressure information measurement device according to a preferred embodiment of the present invention, the tightening force detection mechanism may detect rotation torque applied to the winding roller at a time of driving and rotating the winding roller by the electric motor, thereby detecting the tightening force of the tightening belt over the measurement site.

In the blood pressure information measurement device according to a preferred embodiment of the present invention, the expanding/contracting mechanism preferably starts a pressurization operation of the fluid bag to measure blood pressure information after stopping the winding operation of the tightening belt by the winding roller.

In the blood pressure information measurement device according to a preferred embodiment of the present invention, the tightening length adjustment mechanism preferably starts a feeding operation of the tightening belt by the winding roller after finishing a measurement operation for measuring blood pressure information.

In the blood pressure information measurement device according to a preferred embodiment of the present invention, the cuff main body portion preferably further includes a flexible member that is elastically deformable in the radial direction on the outer side of the fluid bag and on the inner side of the tightening belt.

According to a preferred embodiment of the present invention, in the sphygmomanometer in which the cuff and the main body are separated from each other, the cuff can be easily attached to the measurement site, and reliable winding of the cuff around the measurement site can be repeated in every measurement. Therefore, the blood pressure information measurement device has favorable usability while being capable of precisely and stably measuring the blood pressure information.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a view showing an outer appearance structure of a sphygmomanometer according to a first preferred embodiment of the present invention.

FIG. 2 is a functional block configuration diagram of the sphygmomanometer according to the first preferred embodiment of the present invention.

FIG. 3 is a perspective view showing a detailed structure of a cuff of the sphygmomanometer according to the first preferred embodiment of the present invention.

FIG. 4 is a sectional view along the line IV-IV in FIG. 3 showing a non-attachment state of the cuff.

FIG. 5 is a schematic top view showing a configuration of a tightening length adjustment mechanism of the sphygmomanometer according to the first preferred embodiment of the present invention.

FIG. 6 is a flowchart showing processing procedures of the sphygmomanometer according to the first preferred embodiment of the present invention.

FIG. 7 is a timing chart showing in chronological order operation situations and operation states of respective portions of the sphygmomanometer according to the first preferred embodiment of the present invention.

FIG. 8 is a schematic view for describing an attachment task of attaching to an upper arm the cuff of the sphygmomanometer according to the first preferred embodiment of the present invention.

FIG. 9 is a sectional view of an attachment state where the cuff of the sphygmomanometer according to the first preferred embodiment of the present invention is attached to the upper arm.

FIG. 10 is a functional block configuration diagram of a sphygmomanometer according to a second preferred embodiment of the present invention.

FIG. 11 is a flowchart showing processing procedures of the sphygmomanometer according to the second preferred embodiment of the present invention.

FIG. 12 is a timing chart showing in chronological order operation situations and operation states of respective components of the sphygmomanometer according to the second preferred embodiment of the present invention.

### DETAILED DESCRIPTION OF THE PREFERRED EMBODIMENTS

Preferred embodiments of the present invention will be hereinafter described in detail with reference to the drawings. It should be noted that in the preferred embodiments described hereinafter, description will be made exemplifying a so-called upper arm type sphygmomanometer capable of measuring a systolic blood pressure value and a diastolic blood pressure value as a blood pressure information measurement device as examples only. It should be noted that in the sphygmomanometer according to the preferred embodiments shown hereinafter, a tightening operation of a cuff separated from a main body over an upper arm, a measurement operation of a blood pressure value performed after the tightening operation, and a tightening cancellation operation of the cuff over the upper arm performed after the measurement operation preferably are automatically continuously performed.

### First Preferred Embodiment

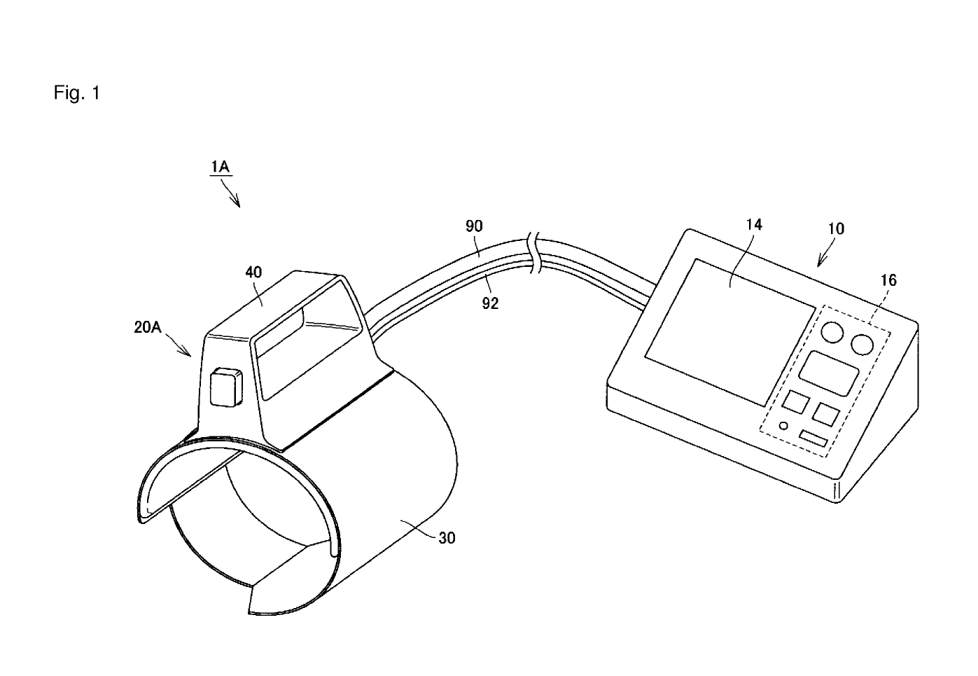

FIG. 1 is a view showing an outer appearance structure of a sphygmomanometer according to a first preferred embodiment of the present invention. Firstly, with reference to FIG. 1, the outer appearance structure of a sphygmomanometer 1A according to the present preferred embodiment will be described.

As shown in FIG. 1, the sphygmomanometer 1A according to the present preferred embodiment is provided with a main body 10, a cuff 20A, an air tube 90, and a connection cable 92. The main body 10 has a box shaped casing, and a display unit 14 and an operation unit 16 are provided on an upper surface thereof. The main body 10 is mounted on a surface of a table or the like to be used at a time of measurement. The cuff 20A has a tubular cuff main body portion 30 including a hollow opening portion into which an upper arm is insertable in the axial direction, and a gripping portion 40 provided on an outer peripheral surface of this cuff main body portion 30. The cuff 20A is attached to the upper arm to be used at a time of measurement. The air tube 90 and the connection cable 92 respectively connect the main body 10 and the cuff 20A separated from each other.

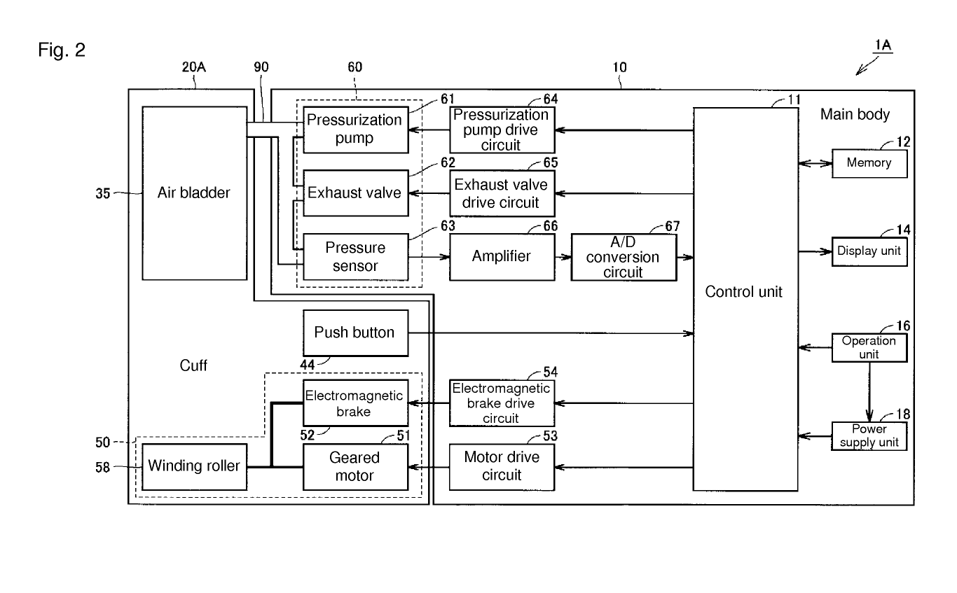

FIG. 2 is a functional block configuration diagram of the sphygmomanometer according to the present preferred embodiment. Next, with reference to FIG. 2, a functional block configuration of the sphygmomanometer 1A according to the present preferred embodiment will be described.

As shown in FIG. 2, the main body 10 includes a control unit 11, a memory unit 12, a power supply unit 18, a motor drive circuit 53, an electromagnetic brake drive circuit 54, a pressurization pump 61, an exhaust valve 62, a pressure sensor 63, a pressurization pump drive circuit 64, an exhaust valve drive circuit 65, an amplifier 66, and an A/D (Analog/Digital) conversion circuit 67 in addition to the display unit 14 and the operation unit 16. Meanwhile, the cuff 20A includes an air bladder 35, a push button 44, a geared motor 51, an electromagnetic brake 52, and a winding roller 58.

The control unit 11 includes, for example, a CPU (Central Processing Unit) that is programmed and arranged to control the entire sphygmomanometer 1A. The memory unit 12 includes, for example, a ROM (Read-Only Memory) and a RAM (Random-Access Memory) to store a program the causes the control unit 11 and the like to execute procedures to measure a blood pressure value and store a measurement result and the like. The display unit 14 includes, for example, a LCD (Liquid Crystal Display) arranged to display the measurement result and the like. The operation unit 16 is arranged to receive an operation of a subject or the like and input this command from the outside to the control unit 11 and the power supply unit 18. The power supply unit 18 is arranged to supply electric power as a power supply to the control unit 11.

The control unit 11 inputs control signals to drive the geared motor 51, the electromagnetic brake 52, the pressurization pump 61 and the exhaust valve 62 to the motor drive circuit 53, the electromagnetic brake drive circuit 54, the pressurization pump drive circuit 64 and the exhaust valve drive circuit 65, and inputs the blood pressure value as the measurement result to the memory unit 12 and the display unit 14. The control unit 11 acquires the blood pressure value of the subject based on a pressure value detected by the pressure sensor 63. The blood pressure value acquired by this control unit 11 is inputted to the memory unit 12 and the display unit 14 as the measurement result. It should be noted that the sphygmomanometer 1A may separately include an output unit arranged to output the blood pressure value as the measurement result to an external device (such as a PC (Personal Computer) and a printer, for example). For example, a serial communication line, a writing device to various recording media and the like can be utilized as the output unit.

The motor drive circuit 53 controls an operation of the geared motor 51 based on the control signal inputted from the control unit 11. The electromagnetic brake drive circuit 54 controls an operation of the electromagnetic brake 52 based on the control signal inputted from the control unit 11. The pressurization pump drive circuit 64 controls an operation of the pressurization pump 61 based on the control signal inputted from the control unit 11. The exhaust valve drive circuit 65 controls an open/close operation of the exhaust valve 62 based on the control signal inputted from the control unit 11.

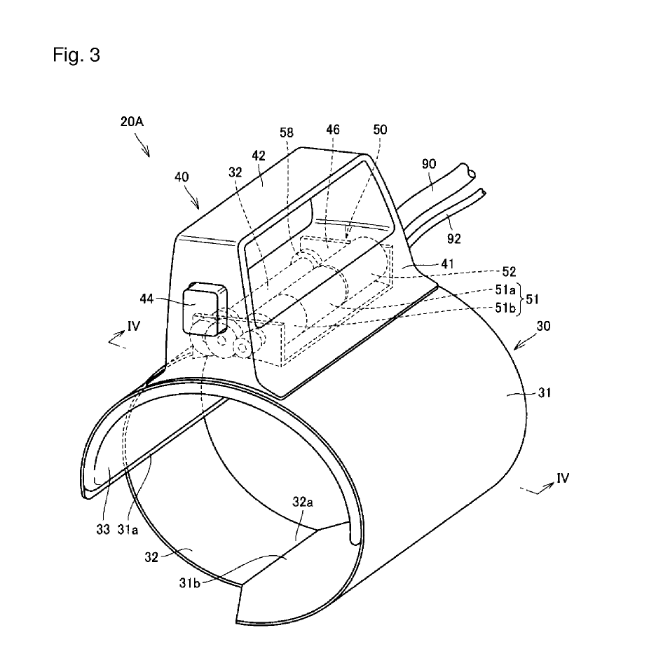

The air bladder 35 is a fluid bag arranged to compress the upper arm in an attachment state, which is connected to an air system component 60 described later via the air tube 90. The geared motor 51, the electromagnetic brake 52 and the winding roller 58 correspond to a tightening length adjustment mechanism 50 to variably adjust tightening lengths of the tightening belts 31, 32 over the upper arm described later (refer to FIG. 3 and the like). Electric connection between the geared motor 51 and the motor drive circuit 53 and electric connection between the electromagnetic brake 52 and the electromagnetic brake drive circuit 54 are respectively performed by the connection cable 92 (refer to FIG. 1).

The geared motor 51 is an electric motor arranged to drive and rotate the winding roller 58 in the forward direction and the backward direction, and an operation thereof is controlled by the motor drive circuit 53. The electromagnetic brake 52 is a brake arranged to apply a braking force to the winding roller 58, and an operation thereof is controlled by the electromagnetic brake drive circuit 54. The winding roller 58 is a member arranged to wind and feed the tightening belts 31, 32 described later (refer to FIG. 3 and the like). The push button 44 corresponds to a winding operation unit arranged to receive the command of the subject to start a winding operation by the tightening length adjustment mechanism 50 defined by the geared motor 51, the electromagnetic brake 52 and the winding roller 58.

The pressurization pump 61 supplies the air into an inner cavity of the air bladder 35, and an operation thereof is controlled by the pressurization pump drive circuit 64. The exhaust valve 62 maintains pressure inside the air bladder 35 (hereinafter, also referred to as the “cuff pressure”) and opens space inside the air bladder 35 to the outside, and an operation thereof is controlled by the exhaust valve drive circuit 65. The pressure sensor 63 inputs an output value corresponding to the pressure inside the air bladder 35 to the amplifier 66. The amplifier 66 amplifies and inputs an output value of the pressure sensor 63 to the A/D conversion circuit 67. The A/D conversion circuit 67 converts an analog signal inputted from the amplifier 66 into a digital signal and inputs the signal to the control unit 11. It should be noted that among these constituent elements, the pressurization pump 61, the exhaust valve 62 and the pressure sensor 63 preferably correspond to the air system component 60, and particularly the pressurization pump 61 and the exhaust valve 62 correspond to an expanding/contracting mechanism arranged to expand and contract the air bladder 35.

In the sphygmomanometer 1A according to the present preferred embodiment, the air bladder 35 and the air system component 60 preferably are utilized as a tightening force detection mechanism arranged to detect a tightening force of the tightening belts over the upper arm, and a detail thereof will be described later.

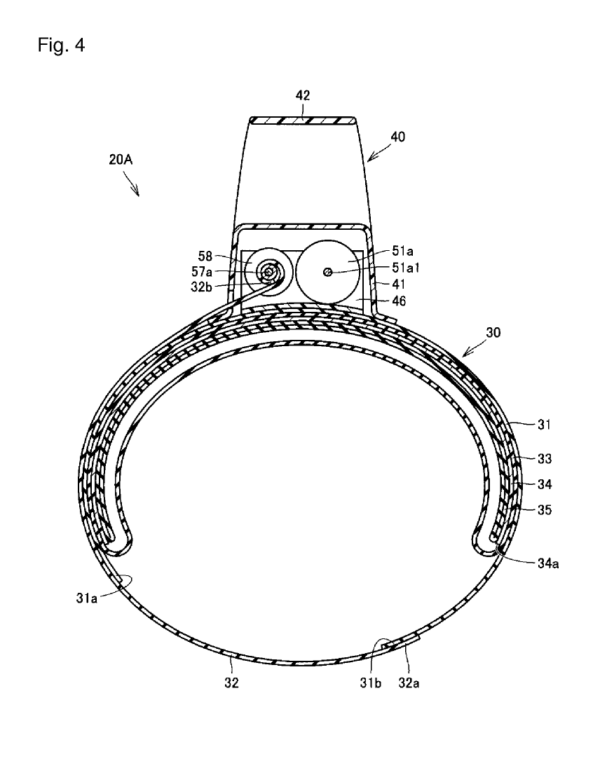

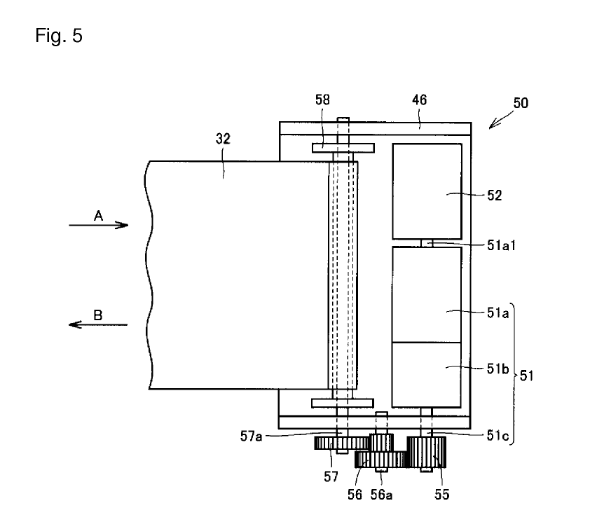

FIG. 3 is a perspective view showing a detailed structure of the cuff of the sphygmomanometer according to the present preferred embodiment, and FIG. 4 is a sectional view along the line IV-IV in FIG. 3 showing a non-attachment state of the cuff. FIG. 5 is a schematic top view showing a configuration of the tightening length adjustment mechanism of the sphygmomanometer according to the present preferred embodiment. Next, with reference to FIGS. 3 to 5, a detailed structure of the cuff 20A of the sphygmomanometer 1A according to the present preferred embodiment will be described.

As shown in FIG. 3, the cuff 20A includes the tubular cuff main body portion 30 to be attached to the upper arm, and the gripping portion 40 provided on the outer peripheral surface of this cuff main body portion 30. The gripping portion 40 includes a base portion 41 attached to the cuff main body portion, and a grip 42 serving as a part to be gripped by a hand at a time of attachment. The cuff main body portion 30 preferably has a tubular shape into which the upper arm is insertable in the axial direction. The gripping portion 40 is fixed to the cuff main body portion 30 so that the grip 42 extends in a direction parallel or substantially parallel to the axial direction of the tubular cuff main body portion 30. The tightening length adjustment mechanism 50 is arranged at a position on the outer peripheral surface of the cuff main body portion 30 and inside the base portion 41 of the gripping portion 40. The push button 44 is provided at a predetermined position of the gripping portion 40.

As shown in FIGS. 3 and 4, the cuff main body portion 30 is provided mainly with the annularly wrapped tightening belts 31, 32, an outer package cover 33 attached to the inner side of the tightening belt 31, and a curler 34 and the air bladder 35 accommodated inside the outer package cover 33. The tightening belts 31, 32 are preferably defined by members such as clothes having no substantial stretchability in the circumferential direction, and include the first tightening belt 31 having a relatively wide width and the second tightening belt 32 having a relatively narrow width, which is coupled to this first tightening belt 31.

The first tightening belt 31 includes a band shaped member including one end 31a and the other end 31b in the circumferential direction. The gripping portion 40 is attached to a predetermined position on an outer peripheral surface thereof, and the outer package cover 33 is attached onto an inner peripheral surface thereof. The second tightening belt 32 includes one end 32a and the other end 32b in the circumferential direction, and the one end 32a is coupled to the other end 31b of the first tightening belt 31. A portion of the second tightening belt 32 close to the other end 32b overlies the outer peripheral side of a portion of the first tightening belt 31 close to the one end 31a. The other end 32b of the second tightening belt 32 is fixed to the winding roller 58 arranged in the gripping portion 40 attached onto the outer peripheral surface of the first tightening belt 31. Thereby, the first tightening belt 31 and the second tightening belt 32 function as one annular member, so that the cuff main body portion 30 including the hollow opening portion is provided.

Circumferential lengths of the first tightening belt 31 and the second tightening belt 32 coupled to each other are variably adjusted by the tightening length adjustment mechanism 50 described later. With long circumferential lengths of the first tightening belt 31 and the second tightening belt 32 coupled to each other, the cuff main body portion 30 is in an extended diameter state (the diameter thereof is extended). With short circumferential lengths, the cuff main body portion 30 is in a reduced diameter state (the diameter is reduced).

The outer package cover 33 includes, for example, a member such as a cloth made of a low friction material having stretchability, and attached onto the inner peripheral surface of the first tightening belt 31. In more detail, an outer peripheral surface of the outer package cover 33 is joined to the inner peripheral surface of the first tightening belt 31 by adhesion, welding or the like, so that the outer package cover 33 is fixed to the first tightening belt 31.

The curler 34 accommodated in the outer package cover 33 preferably includes a flexible member formed by injection molding a resin material such as polypropylene as a base material. In more detail, the curler 34 includes an annular curved elastic plate having a cut 34a extending along the axial direction at a predetermined position in the circumferential direction, and formed in a C shape or a U shape when the curler is cut along a plane that is perpendicular or substantially perpendicular to the axial direction. The curler 34 maintains its own annular form and is elastically deformable in the radial direction. Therefore, the curler 34 has a largely extended diameter in the extended diameter state and on the other hand has a narrowed diameter in the reduced diameter state. It should be noted that when the cuff main body portion 30 is in the extended diameter state, the cuff main body portion 30 is largely extended by the elastic force of the curler 34. Therefore, the upper arm is easily put in and drawn out of the inside of the hollow opening portion of the cuff main body portion 30.

The air bladder 35 preferably includes a bag shaped member capable of being expanded and contracted and including, for example, two overlapping resin films that include peripheral edges thereof joined to each other via welding, for example. The inner cavity of the air bladder 35 is connected to the air tube 90 via a nipple (not shown). The inner cavity of the air bladder 35 is pressurized and depressurized by the pressurization pump 61 and the exhaust valve 62 provided in the main body 10, so that the air bladder 35 is expanded or contracted.

As shown in FIGS. 3 to 5, the tightening length adjustment mechanism 50 preferably includes the geared motor 51, the electromagnetic brake 52 and the winding roller 58. The geared motor 51, the electromagnetic brake 52 and the winding roller 58 are respectively assembled to a support frame 46 arranged at a position on the outer peripheral surface of the cuff main body portion 30 and inside the base portion 41 of the gripping portion 40. The support frame 46 is, for example, fixed onto the outer peripheral surface of the first tightening belt 31. Gears 55, 56, 57 serving as a power transmission mechanism are assembled at predetermined positions of the support frame 46.

The geared motor 51 is a motor provided with a reducer and includes a motor unit 51a, a reducer unit 51b and an output shaft 51c. The gear 55 is fixed to the output shaft 51c of the geared motor 51. The electromagnetic brake 52 is arranged adjacent to the geared motor 51 on an end of the geared motor 51 in the axial direction opposite to the side where the output shaft 51c is positioned. The electromagnetic brake 52 fixedly supports a rotation shaft 51a1 of the motor unit 51a so as to apply braking force to the rotation shaft 51a1.

The winding roller 58 is fixed to a shaft 57a axially supported on the support frame 46 and driven and rotated by rotation of the shaft 57a. The other end 32b of the second tightening belt 32 is fixed to the winding roller 58. The gear 57 is fixed to the shaft 57a to which the winding roller 58 is fixed. The gear 56 is fixed to a shaft 56a axially supported on the support frame 46. The gear 56 is respectively meshed with the gear 55 and the gear 57, and transmits rotation force generated in the output shaft 51c of the geared motor 51 to the winding roller 58. It should be noted that outer diameters and the number of teeth of these gears 55, 56, 57 are respectively adjusted, and the gears also function as a reducer as well as the reducer unit 51b of the geared motor 51.

Next, with reference to FIG. 5, an operation of the tightening length adjustment mechanism 50 of the cuff 20A of the sphygmomanometer 1A according to the present preferred embodiment will be described. In the sphygmomanometer 1A according to the present preferred embodiment, as described above, the tightening operation of the cuff 20A over the upper arm, the measurement operation of the blood pressure value performed after the tightening operation, and the tightening cancellation operation of the cuff 20A over the upper arm performed after the measurement operation are automatically continuously performed. The tightening operation of the cuff 20A over the upper arm and the tightening cancellation operation of the cuff 20A over the upper arm are respectively performed by the winding operation of the tightening belts 31, 32 by the tightening length adjustment mechanism 50 and a feeding operation of the tightening belts 31, 32 by the tightening length adjustment mechanism 50 described later.

With reference to FIG. 5, in a state where the geared motor 51 is driven and rotated in the forward direction, the output shaft 51c of the geared motor 51 is rotated in the forward direction, the rotation force thereof is transmitted to the shaft 57a via the gears 55, 56, 57, and the winding roller 58 is rotated in the forward direction. Since the winding roller 58 is rotated in the forward direction, the second tightening belt 32 with the other end 32b fixed to the winding roller 58 is wound in the arrow A direction in the figure by the winding roller 58. By the winding operation of the second tightening belt 32 by this winding roller 58, the tightening lengths of the tightening belts 31, 32 are reduced against the elastic force of the curler 34, and the diameter of the hollow opening portion of the cuff main body portion 30 is gradually reduced. That is, by the winding operation, the tightening operation of the cuff 20A over the upper arm is realized. It should be noted that at a time of driving and rotating the geared motor 51 in the forward direction, the electromagnetic brake 52 does not fixedly support the rotation shaft 51a1 of the motor unit 51a of the geared motor 51. The motor unit 51a is driven while an operation thereof is not limited.

Meanwhile, in a state where the geared motor 51 is driven and rotated in the backward direction, the output shaft 51c of the geared motor 51 is rotated in the backward direction, the rotation force thereof is transmitted to the shaft 57a via the gears 55, 56, 57, and the winding roller 58 is rotated in the backward direction. Since the winding roller 58 is rotated in the backward direction, a portion of the second tightening belt 32 wound by the winding roller 58 is fed in the arrow B direction in the figure from the winding roller 58. The tightening lengths of the tightening belts 31, 32 are increased by the feeding operation of the second tightening belt 32 by this winding roller 58. At that time, the diameter of the hollow opening portion of the cuff main body portion 30 is gradually extended based on the elastic force of the curler 34. That is, by the feeding operation, the tightening cancellation operation of the cuff 20A over the upper arm is realized. It should be noted that at a time of driving and rotating the geared motor 51 in the backward direction, the electromagnetic brake 52 does not fixedly support the rotation shaft 51a1 of the motor unit 51a of the geared motor 51. The motor unit 51a is driven while the operation thereof is not limited.

In a state in which the geared motor 51 is not driven and rotated in the forward direction or the backward direction, that is, at a time of stopping the geared motor 51, the rotation shaft 51a1 of the motor unit 51a of the geared motor 51 is fixedly supported by the electromagnetic brake 52. In the above-described state, the braking force by the electromagnetic brake 52 is applied to the winding roller 58 via the rotation shaft 51a1 of the motor unit 51a, the reducer unit 51b, the output shaft 51c, the gears 55, 56, 57 and the shaft 57a, so that a rotation operation of the winding roller 58 is limited. Therefore, in the above-described state, the winding and feeding operations of the second tightening belt 32 by the winding roller 58 are both stopped, so that the diameter of the hollow opening portion of the cuff main body portion 30 is maintained to be constant.

Next, the tightening force detection mechanism provided in the sphygmomanometer 1A according to the present preferred embodiment will be described. The tightening force detection mechanism detects the tightening force of the cuff 20A over the upper arm during the tightening operation of the cuff 20A so that a tightening state of the cuff 20A over the upper arm is optimal.

As described above, in the sphygmomanometer 1A according to the present preferred embodiment, the tightening force detection mechanism is preferably defined by the air bladder 35 and the air system component 60. This tightening force detection mechanism is a mechanism arranged to detect the tightening force of the tightening belts 31, 32 over the upper arm, and capture the tightening force as an internal pressure of the air bladder 35.

Specifically, in the sphygmomanometer 1A according to the present preferred embodiment, a predetermined amount of the air is injected into the air bladder 35 by the pressurization pump 61 before driving the tightening length adjustment mechanism 50 so as to reduce the diameter of the hollow opening portion of the cuff main body portion 30, and the internal pressure of the air bladder 35 to be nipped between the tightening belts 31, 32 with the tightening lengths reduced in accordance with the drive of the tightening length adjustment mechanism 50 and the upper arm is detected by the pressure sensor 63, so that the tightening force of the tightening belts 31, 32 over the upper arm is detected based on the detected internal pressure of the air bladder 35.

The control unit 11 monitors the internal pressure of the air bladder 35 during the tightening operation by the tightening length adjustment mechanism 50, stops the operation of the geared motor 51 at the time point when the internal pressure becomes a predetermined pressure value, and at the same time operates the electromagnetic brake 52 so as to stop the rotation of the winding roller 58. As described above, the tightening state of the cuff 20A over the upper arm is thus optimal.

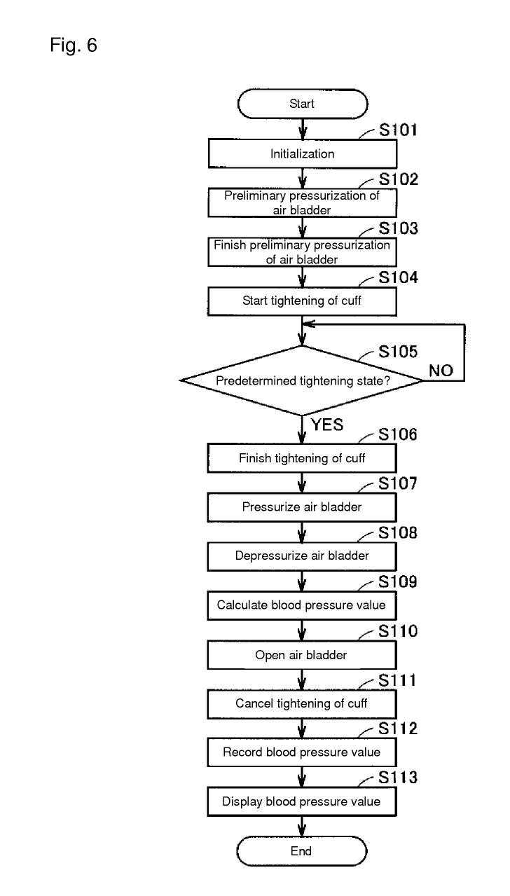

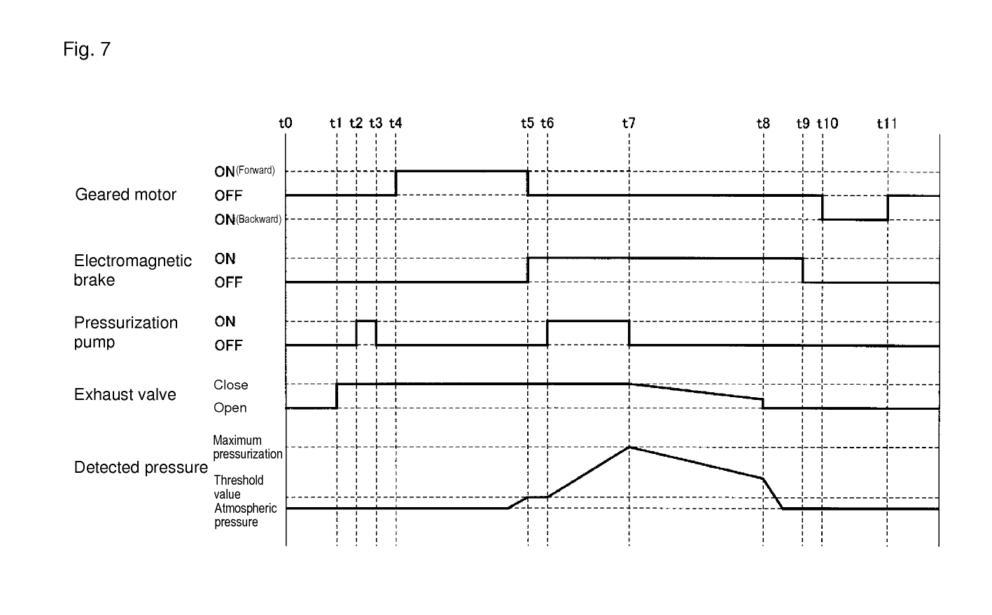

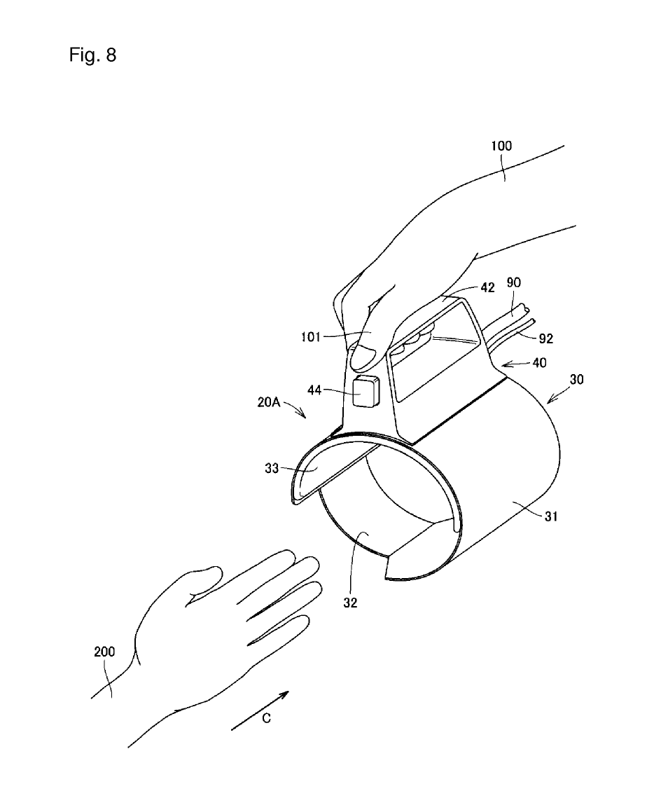

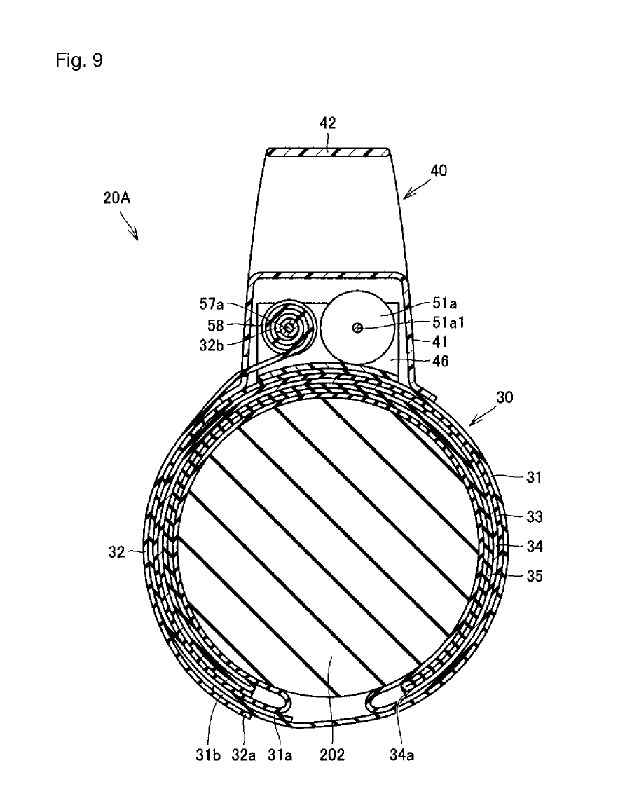

FIG. 6 is a flowchart showing processing procedures of the sphygmomanometer according to the present preferred embodiment, and FIG. 7 is a timing chart showing in chronological order operation situations and operation states of parts of the sphygmomanometer according to the present preferred embodiment. FIG. 8 is a schematic view for describing an attachment task for attaching to the upper arm the cuff of the sphygmomanometer according to the present preferred embodiment, and FIG. 9 is a sectional view of an attachment state where the cuff of the sphygmomanometer according to the present preferred embodiment is attached to the upper arm. Next, with reference to FIGS. 6 to 9, the processing procedures of the sphygmomanometer 1A according to the present preferred embodiment will be described with the operation situations and the operation states of the components of the sphygmomanometer 1A or the attachment task of the cuff 20A and the state after the attachment of the cuff 20A. It should be noted that a program according to the flowchart shown in FIG. 6 is preliminarily stored in the memory unit 12 shown in FIG. 2, and the control unit 11 reads out and executes this program from the memory unit 12, so that processing thereof is performed.

Firstly, as shown in FIG. 6, when the subject operates the operation unit 16 of the sphygmomanometer 1A and inputs a command to turn ON power, the electric power as the power supply is supplied from the power supply unit 18 to the control unit 11, so that the control unit 11 is driven and initialization of the sphygmomanometer 1A is performed (Step S101). As shown in FIG. 7, at time t0 when the initialization of the sphygmomanometer 1A is performed, the geared motor 51, the electromagnetic brake 52 and the pressurization pump 61 are all in a OFF state in which the operations are stopped, the exhaust valve 62 is opened so as to provide communication between the space inside the air bladder 35 and the outside and equalize the cuff pressure to an atmospheric pressure, and the cuff pressure detected by the pressure sensor 63 indicates the same value as the atmospheric pressure.

Next, as shown in FIG. 8, the subject grips the grip 42 of the cuff 20A with a right hand 100, and inserts a left hand 200 which is different from the right hand 100 gripping the grip 42 into the hollow opening portion of the cuff main body portion 30 of the cuff 20A in the arrow C direction in the figure. Then the subject moves the cuff 20A to the upper arm of the left hand 200, and pushes the push button 44 provided in the grip 42 with a thumb 101 of the right hand 100 which is the hand for gripping the grip 42 while maintaining a state where the cuff is placed on the upper arm of the left hand 200.

As shown in FIG. 6, the control unit 11 which received the pushing of the push button 44 by the subject performs preliminary pressurization of the air bladder 35 (Step S102). Specifically, as shown in FIG. 7, the control unit 11 closes the exhaust valve 62 so as not to provide communication between the space inside the air bladder 35 and the outside at time t1 when the push button 44 is pushed, and successively starts driving the pressurization pump 61 so as to inject the air into the air bladder 35 at time t2. The control unit 11 stops driving the pressurization pump 61 at time t3 after elapse of a predetermined period. A period when the pressurization pump 61 is driven is a period required to inject a predetermined amount of the air into the space inside the air bladder 35. As described above, the preliminary pressurization of the air bladder 35 is finished (Step S103).

Next, as shown in FIG. 6, the control unit 11 starts the tightening operation of the cuff 20A over the upper arm (Step S104). At that time, the control unit 11 determines whether or not the tightening of the cuff 20A over the upper arm is in a predetermined tightening state (Step S105). When the tightening is not in the predetermined tightening state (NO in Step S105), the control unit continues the tightening operation of the cuff 20A over the upper arm. When the tightening is in the predetermined tightening state (YES in Step S105), the control unit stops the tightening operation of the cuff 20A over the upper arm (Step S106).

Specifically, as shown in FIG. 7, the control unit 11 drives and rotates the geared motor 51 in the forward direction at time t4 so as to start the winding operation of the second tightening belt 32 by the winding roller 58. The control unit 11 detects the cuff pressure of the air bladder 35 by the pressure sensor 63 during the winding operation, stops driving and rotating the geared motor 51 in the forward direction at time t5 when the detected cuff pressure reaches a preliminarily fixed threshold value, and at the same time drives the electromagnetic brake 52 so as to stop the rotation of the winding roller 58. The threshold value is preliminarily determined based on the tightening force of the cuff 20A over the upper arm which is suitable for measuring the blood pressure value.

As shown in FIG. 9, in a state where the cuff 20A is tightened over the upper arm 202 with optimal tightening force, the air bladder 35 is reliably pressed onto the upper arm 202 by the tightening belts 31, 32. Therefore, the air bladder 35 is expanded in the following measurement operation, so that the upper arm 202 is reliably compressed by the cuff 20A. Thus, restriction in blood supply to an artery positioned inside the upper arm 202 can be reliably performed.

Next, as shown in FIG. 6, the control unit 11 starts pressurization of the air bladder 35 to measure the blood pressure value (Step S107). Specifically, as shown in FIG. 7, the control unit 11 drives the pressurization pump 61 at time t6 and increases the cuff pressure so as to pressurize the air bladder 35 to obtain a predetermined cuff pressure.

Next, as shown in FIG. 6, the control unit 11 starts slow depressurization of the air bladder 35 to measure the blood pressure value (Step S108). Specifically, as shown in FIG. 7, the control unit 11 stops driving the pressurization pump 61 at time t7 when the pressure sensor 63 detects that the internal pressure of the air bladder 35 reaches a predetermined internal pressure, and then gradually opens the exhaust valve 62 while controlling an open amount of the exhaust valve 62. At that time, the control unit 11 acquires a change in the cuff pressure detected by the pressure sensor 63.

Next, as shown in FIG. 6, the control unit 11 calculates the blood pressure value based on the change in the cuff pressure obtained in the slow depressurization process (Step S109). Successively, the control unit 11 opens the air bladder 35 (Step S110), and also performs the tightening cancellation operation of the cuff 20A over the upper arm 202 (Step S111). Specifically, as shown in FIG. 7, the control unit 11 completely opens the exhaust valve 62 at time t8 when the calculation of the blood pressure value is finished so as to exhaust the air in the air bladder 35 to the outside, then stops the operation of the electromagnetic brake 52 at time t9, and drives and rotates the geared motor 51 in the backward direction at time t10 so as to feed the second tightening belt 32 from the winding roller 58. Then, the control unit 11 stops driving the geared motor 51 at time t11 when the second tightening belt 32 is completely fed out from the winding roller 58. The stoppage of the drive of the geared motor 51 is controlled based on time here. However, a detector such as an optical sensor may be provided in the cuff main body portion 30 so as to detect that a terminal of the second tightening belt 32 is fed out from the winding roller 58, thereby controlling to stop the drive of the geared motor 51.

Next, as shown in FIG. 6, the control unit 11 outputs the blood pressure value obtained in Step S109 to the memory unit 12 and the display unit 14 and the blood pressure value is stored in the memory unit 12 as the measurement result (Step S112). The blood pressure value as the measurement result is displayed in the display unit 14 (Step S113). The display unit 14 displays the systolic blood pressure value and the diastolic blood pressure value, for example, as numerical values. The sphygmomanometer 1A is in a standby state after recording and displaying these blood pressure values, and stops supply of the electric power as the power supply upon input of a command to turn OFF power by the subject with the operation unit 16.

With the sphygmomanometer 1A as described above, by highly simple operations of gripping the grip 42 provided in the cuff 20A with the right hand 100 which is different from the left hand 200 to which the cuff 20A is attached, inserting the left hand 200 to which the cuff 20A is attached into the hollow opening portion of the cuff 20A in that state so as to place the cuff 20A on the upper arm 202, and then pushing the push button 44 provided in the grip 42, the tightening operation of the cuff 20A over the upper arm 202 can be consequently automatically performed. Therefore, the cuff 20A can be highly easily attached to the upper arm 202 serving as a measurement site.

In the sphygmomanometer 1A according to the present preferred embodiment, in a time of tightening the upper arm 202 by using the tightening belts 31, 32, the tightening force of the cuff 20A over the upper arm 202 is detected by using the tightening force detection mechanism, and the state where the tightening force is optimal is maintained by using the tightening length adjustment mechanism 50. Therefore, reliable winding of the cuff 20A over the upper arm 202 can be repeated in every measurement.

Therefore, by adopting the above configuration, in the sphygmomanometer 1A in which the cuff 20A and the main body 10 are separated from each other, the cuff 20A can be easily attached to the upper arm 202, and reliable winding of the cuff 20A over the upper arm 202 can be repeated in every measurement. As a result, the sphygmomanometer can have favorable usability while being capable of precisely and stably measuring the blood pressure value.

In the sphygmomanometer 1A according to the present preferred embodiment, not only the attachment task of the cuff 20A but also the measurement task of the blood pressure value performed after that and the tightening cancellation task of the cuff 20A over the upper arm 202 performed after the measurement task are all automatically continuously performed. Therefore, by adopting the above configuration, the sphygmomanometer can have highly excellent convenience capable of attaching the cuff 20A, measuring the blood pressure value and detaching the cuff 20A with a so-called single touch operation.

Further, in the sphygmomanometer 1A according to the present preferred embodiment, the geared motor 51, the electromagnetic brake 52, the winding roller 58 and the like serving as the tightening length adjustment mechanism 50 are accommodated in the base portion 41 of the gripping portion 40. Thus, since the cuff 20A can be downsized and made extremely compact, an effect of not increasing size of the cuff 20A can be obtained.

### Second Preferred Embodiment

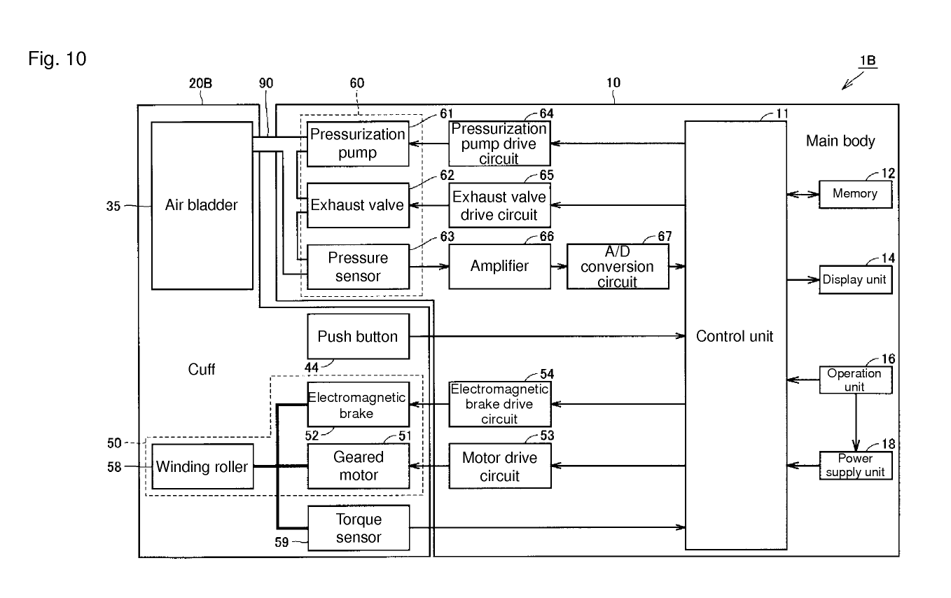

FIG. 10 is a functional block configuration diagram of a sphygmomanometer according to a second preferred embodiment of the present invention. Firstly, with reference to FIG. 10, a functional block configuration of a sphygmomanometer 1B according to the present preferred embodiment will be described. It should be noted that the sphygmomanometer 1B according to the present preferred embodiment is the same as the sphygmomanometer 1A according to the first preferred embodiment in terms of an outer appearance structure, and major components of the functional block configuration thereof are also common to the first preferred embodiment. Therefore, the same reference numerals are given to similar components to the first preferred embodiment in the figures, and the description thereof will not be repeated.

In the sphygmomanometer 1A according to the first preferred embodiment, the air bladder 35 and the air system component 60 preferably are utilized as the tightening force detection mechanism to detect the tightening force of the tightening belts 31, 32 over the upper arm 202, the tightening force is captured as the internal pressure of the air bladder 35. Meanwhile, in the sphygmomanometer 1B according to the present preferred embodiment, a torque sensor arranged to detect a rotation torque applied to the winding roller 58 around which the second tightening belt 32 is wound is preferably used as the tightening force detection mechanism to detect the tightening force of the tightening belts 31, 32 over the upper arm 202, and thereby the tightening force is captured as the rotation torque applied to the winding roller 58.

As shown in FIG. 10, in the sphygmomanometer 1B according to the present preferred embodiment, a torque sensor 59 is preferably provided in the cuff 20B. The torque sensor 59 is arranged to detect the rotation torque applied to the winding roller 58, and, for example, attached to the shaft 57a to which the winding roller 58 is fixed (refer to FIG. 5).

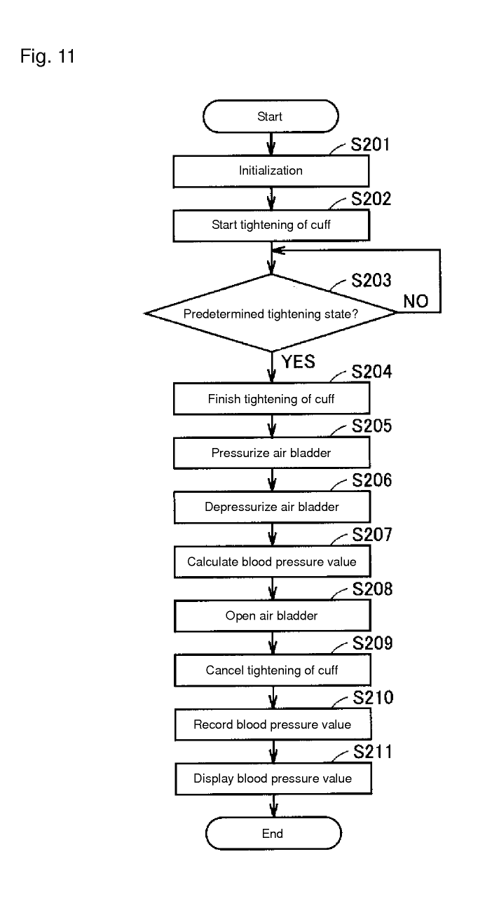

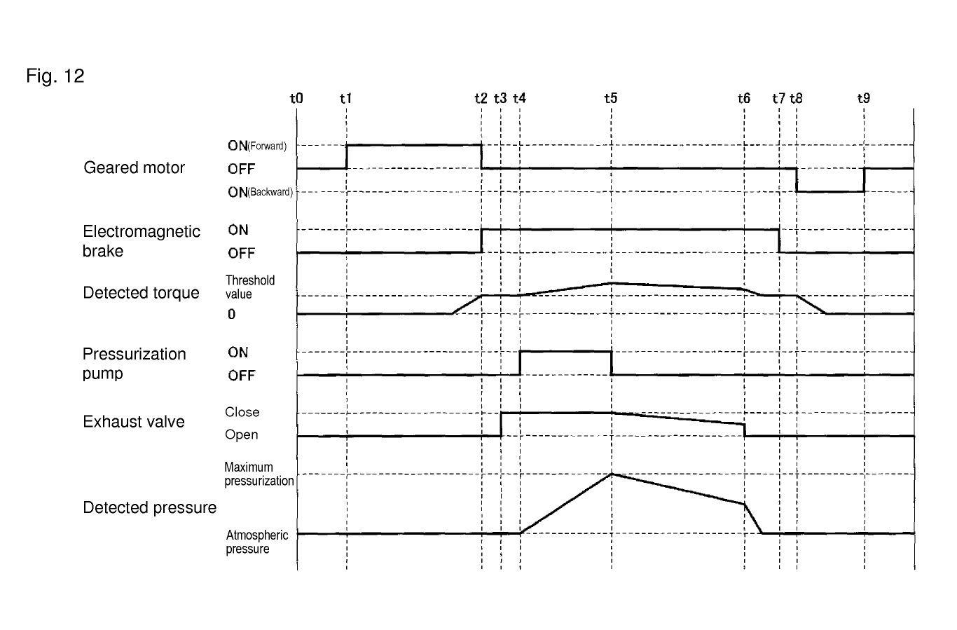

FIG. 11 is a flowchart showing processing procedures of the sphygmomanometer according to the present preferred embodiment, and FIG. 12 is a timing chart showing in chronological order operation situations and operation states of components of the sphygmomanometer according to the present preferred embodiment. Next, with reference to FIGS. 11 and 12, the processing procedures of the sphygmomanometer 1B according to the present preferred embodiment will be described with the operation situations and the operation states of the components of the sphygmomanometer 1B. It should be noted that a program according to the flowchart shown in FIG. 11 preferably is preliminarily stored in the memory unit 12 shown in FIG. 10, and the control unit 11 reads out and executes this program from the memory unit 12, so that processing thereof is progressed.

Firstly, as shown in FIG. 11, when the subject operates the operation unit 16 of the sphygmomanometer 1B and inputs the command to turn ON power, the electric power as the power supply is supplied from the power supply unit 18 to the control unit 11, so that the control unit 11 is driven and initialization of the sphygmomanometer 1B is performed (Step S201). As shown in FIG. 12, at time t0 when the initialization of the sphygmomanometer 1B is performed, the geared motor 51, the electromagnetic brake 52 and the pressurization pump 61 are all in the OFF state in which the operations are stopped, the rotation torque detected by the torque sensor 59 is substantially zero, the exhaust valve 62 is opened so as to provide communication between the space inside the air bladder 35 and the outside and equalize the cuff pressure to the atmospheric pressure, and the cuff pressure detected by the pressure sensor 63 indicates the same value as the atmospheric pressure.

Next, as in the first preferred embodiment, the subject grips the grip 42 of the cuff 20B with the right hand 100, and inserts the left hand 200 which is different from the right hand 100 gripping the grip 42 into the hollow opening portion of the cuff main body portion 30 of the cuff 20B (refer to FIG. 8). Then the subject moves the cuff 20B to the upper arm of the left hand 200, and pushes the push button 44 provided in the grip 42 with the thumb 101 of the right hand 100 which is the hand for gripping the grip 42 while maintaining the state where the cuff is placed on the upper arm of the left hand 200.

As shown in FIG. 11, the control unit 11 which received the pushing of the pushbutton 44 by the subject starts the tightening operation of the cuff 20B over the upper arm (Step S202). At that time, the control unit 11 determines whether or not the tightening of the cuff 20B over the upper arm is in the predetermined tightening state (Step S203). When the tightening is not in the predetermined tightening state (NO in Step S203), the control unit continues the tightening operation of the cuff 20B over the upper arm. When the tightening is in the predetermined tightening state (YES in Step S203), the control unit stops the tightening operation of the cuff 20B over the upper arm (Step S204).

Specifically, as shown in FIG. 12, the control unit 11 drives and rotates the geared motor 51 in the forward direction at time t1 so as to start the winding operation of the second tightening belt 32 by the winding roller 58. The control unit 11 detects the rotation torque applied to the winding roller 58 during the winding operation by the torque sensor 59, stops driving and rotating the geared motor 51 in the forward direction at time t2 when the detected rotation torque reaches a preliminarily fixed threshold value, and at the same time drives the electromagnetic brake 52 so as to stop the rotation of the winding roller 58. The threshold value is preliminarily determined based on the tightening force of the cuff 20B over the upper arm which is suitable for measuring the blood pressure value.

As in the first preferred embodiment, in a state where the cuff 20B is tightened over the upper arm 202 with optimal tightening force, the air bladder 35 is reliably pressed onto the upper arm 202 by the tightening belts 31, 32 (refer to FIG. 9). Therefore, the air bladder 35 is expanded in the following measurement operation, so that the upper arm 202 is reliably compressed by the cuff 20B. Thus, restriction in blood supply to the artery positioned inside the upper arm 202 can be reliably performed.

Next, as shown in FIG. 11, the control unit 11 starts the pressurization of the air bladder 35 to measure the blood pressure value (Step S205). Specifically, as shown in FIG. 12, the control unit 11 closes the exhaust valve 62 so as not to provide communication between the space inside the air bladder 35 and the outside at time t3, successively starts driving the pressurization pump 61 to inject the air into the air bladder 35 at time t4 and increases the cuff pressure so as to pressurize the air bladder 35 to obtain predetermined cuff pressure.

Next, as shown in FIG. 11, the control unit 11 starts the slow depressurization of the air bladder 35 to measure the blood pressure value (Step S206). Specifically, as shown in FIG. 12, the control unit 11 stops driving the pressurization pump 61 at time t5 when the pressure sensor 63 detects that the internal pressure of the air bladder 35 reaches a predetermined internal pressure, and then gradually opens the exhaust valve 62 while controlling the open amount of the exhaust valve 62. At that time, the control unit 11 acquires the change in the cuff pressure detected by the pressure sensor 63.

Next, as shown in FIG. 11, the control unit 11 calculates the blood pressure value based on the change in the cuff pressure obtained in the slow depressurization process (Step S207). Successively, the control unit 11 opens the air bladder 35 (Step S208), and also performs the tightening cancellation operation of the cuff 20B over the upper arm 202 (Step S209). Specifically, as shown in FIG. 12, the control unit 11 completely opens the exhaust valve 62 at time t6 when the calculation of the blood pressure value is finished so as to exhaust the air in the air bladder 35 to the outside, then stops the operation of the electromagnetic brake 52 at time t7, and drives and rotates the geared motor 51 in the backward direction at time t8 so as to feed out the second tightening belt 32 from the winding roller 58. Then, the control unit 11 stops driving the geared motor 51 at time t9 when the second tightening belt 32 is completely fed out from the winding roller 58.

Next, as shown in FIG. 11, the control unit 11 outputs the blood pressure value obtained in Step S207 to the memory unit 12 and the display unit 14 and the blood pressure value is stored in the memory unit 12 as the measurement result (Step S210). The blood pressure value as the measurement result is displayed in the display unit 14 (Step S211). The display unit 14 displays the systolic blood pressure value and the diastolic blood pressure value, for example, as the numerical values. The sphygmomanometer 1B is in the standby state after recording and displaying these blood pressure values, and stops the supply of the electric power as the power supply upon the input of the command to turn OFF power by the subject with the operation unit 16.

With the sphygmomanometer 1B as described above, the same effects and advantages as achieved by the sphygmomanometer 1A according to the first preferred embodiment can also be obtained. That is, with the sphygmomanometer 1B as described above, in the sphygmomanometer 1B in which the cuff 20B and the main body 10 are separated from each other, the cuff 20B can be easily attached to the upper arm 202, and reliable winding of the cuff 20B over the upper arm 202 can be repeated in every measurement. As a result, the sphygmomanometer can have favorable usability while being capable of precisely and stably measuring the blood pressure value.

It should be noted that although the case that the winding operation unit to start the winding operation of the second tightening belt 32 is defined by the push button 44 was described and illustrated in the sphygmomanometers 1A, 1B in the above first and second preferred embodiments of the present invention, the winding operation unit is not necessarily defined by the push button 44 but may be defined by a sliding type button, a dial type button, a touch sensor, a voice recognition sensor or the like. A position at which the winding operation unit is provided is not limited to a position of the gripping portion 40 where the winding operation unit is operable by the thumb but the winding operation unit may be provided at other positions of the gripping portion 40 or the main body 10.

The case in which the tightening operation of the cuff over the upper arm, the measurement operation of the blood pressure value performed after the tightening operation, and the tightening cancellation operation of the cuff over the upper arm performed after the measurement operation preferably are automatically continuously performed was described and illustrated in the sphygmomanometers 1A, 1B according to the first and second preferred embodiments of the present invention. However, a series of all the operations are not necessarily automatically continuously performed but the operations may be performed in order based on the operation of the operation unit.

The case in which the upper arm of the left hand serves as an attaching site and the right hand is to grip the gripping portion was described and illustrated in the first and second preferred embodiments of the present invention. However, as a matter of course, an upper arm of the right hand may serve as the attaching site and the left hand may be to grip the gripping portion. The so-called upper arm type sphygmomanometer cuff to be attached to the upper arm at a time of measuring the blood pressure value was described and illustrated as the cuff of the sphygmomanometer according to the first and second preferred embodiments. However, the present invention is not particularly limited to this but as a matter of course, may be applied to a so-called wrist type sphygmomanometer cuff to be attached to a wrist in a time of measuring the blood pressure value, a so-called ankle type sphygmomanometer cuff to be attached to an ankle at a time of measuring the blood pressure value or the like.

The case in which the present invention is preferably applied to the sphygmomanometer capable of measuring the systolic blood pressure value and the diastolic blood pressure value was described and illustrated in the first and second preferred embodiments of the present invention. However, the present invention may be applied to a blood pressure information measurement device capable of other blood pressure information rather than the systolic blood pressure value and the diastolic blood pressure value.

As described above, the preferred embodiments disclosed herein are illustrative in all aspects and should not be construed as being restrictive. The technical scope of the present invention is defined by the claims, and meanings equivalent to the claims and all modifications within the scope are intended to be encompassed herein.

What is claimed is:

1. A blood pressure information measurement device, comprising:
a cuff arranged to be attached to a measurement site at a time of measurement; and
a main body, separate from the cuff, and to be placed on a supporting surface for use at the time of measurement; wherein
the cuff includes:
a tubular cuff main body portion including a hollow opening portion into which the measurement site is insertable in an axial direction; and
a gripping portion provided on an outer peripheral surface of the cuff main body portion; wherein
the cuff main body portion includes:
a fluid bag arranged to compress the measurement site;
a tightening belt wrapped around an outer side of the fluid bag; and
a tightening length adjustment mechanism arranged to variably adjust a tightening length of the tightening belt over the measurement site;
the main body includes:
an expanding/contracting mechanism arranged to expand and contract the fluid bag; and
the tightening length adjustment mechanism includes:
a winding roller arranged to wind and feed the tightening belt;
an electric motor arranged to drive and rotate the winding roller in a forward direction and a backward direction; and
a brake arranged to apply a braking force to the winding roller at a time of stopping the electric motor.

2. The blood pressure information measurement device according to claim 1, wherein
the gripping portion includes a base portion attached to the cuff main body portion, and
the tightening length adjustment mechanism is accommodated in the base portion.

3. The blood pressure information measurement device according to claim 1, wherein a winding operation unit arranged to receive a command to start a winding operation of the tightening belt by the winding roller is provided in the gripping portion.

4. The blood pressure information measurement device according to claim 3, wherein the winding operation unit is defined by a push button.

5. The blood pressure information measurement device according to claim 1, further comprising:
a tightening force detection mechanism arranged to detect a tightening force of the tightening belt over the measurement site, wherein
the tightening length adjustment mechanism is arranged to stop a winding operation of the tightening belt by the winding roller when the tightening force detection mechanism detects a predetermined magnitude of the tightening force.

6. The blood pressure information measurement device according to claim 5, wherein the tightening force detection mechanism is arranged to detect an internal pressure of the fluid bag at a time of the winding operation of the tightening belt by the winding roller in a state in which a predetermined amount of fluid is injected into the fluid bag by the expanding/contracting mechanism, so as to detect the tightening force of the tightening belt over the measurement site.

7. The blood pressure information measurement device according to claim 5, wherein the tightening force detection mechanism is arranged to detect a rotation torque applied to the winding roller at a time of driving and rotating the winding roller by the electric motor, so as to detect the tightening force of the tightening belt over the measurement site.

8. The blood pressure information measurement device according to claim 5, wherein the expanding/contracting mechanism is arranged to start a pressurization operation of the fluid bag to measure blood pressure information after stopping the winding operation of the tightening belt by the winding roller.

9. The blood pressure information measurement device according to claim 5, wherein the tightening length adjustment mechanism is arranged to start a feeding operation of the tightening belt by the winding roller after finishing a measurement operation to measure blood pressure information.

10. The blood pressure information measurement device according to claim 1, wherein the cuff main body portion further includes a flexible member that is elastically deformable in a radial direction on the outer side of the fluid bag and on an inner side of the tightening belt.
